(19) United States
(12) Patent Application Publication
(10) Pub. No.: US 2008/0193526 A1
(43) Pub. Date: Aug. 14, 2008

(54) Pharmaceutical Compositions Useful in the Treatment of Pain

(76) Inventors: Anders Pettersson, Uppsala (SE); Thomas Lundqvist, Uppsala (SE)

Correspondence Address: MORGAN LEWIS & BOCKIUS LLP, 1111 PENNSYLVANIA AVENUE NW, WASHINGTON, DC, 20004, US

(21) Appl. No.: 11/885,126

(22) PCT Filed: Mar. 28, 2006
(86) PCT No.: PCT/GB2006/001132

§ 371 Date: Mar. 6, 2008

Related U.S. Application Data
(60) Provisional application No. 60/665,376, filed on Mar. 28, 2005.

(51) Int. Cl. A61K 9/20; A61K 31/197; A61P 25/16; A61K 9/14

(52) U.S. Cl. 424/464; 514/567; 424/489; 424/501

(57) ABSTRACT

There is provided pharmaceutical compositions that are useful for inter alia the treatment of motor fluctuations in patients receiving L-dopa for the treatment of Parkinson's disease comprising a weakly acidic material and a pharmacologically-effective amount of L-dopa, presented in particulate form upon the surfaces of larger carrier particles.

This invention relates to new, fast acting pharmaceutical compositions that are useful in the treatment of Parkinson's disease, which compositions may be administered transmucosally and in particular sublingually.

Parkinson's disease is a disease that seriously affects a sufferer's movement and coordination.

The disease, which is fairly common (affecting approximately 0.15% of the population at any one time) tends to be more prevalent in older people, but can also occur in younger adults.

The parts of the brain that are affected by the onset of Parkinson's include principally the substantia nigra, which is a part of the brain that controls motor function, as well as the nigrostriatal pathways and the locus coeraleus. The presence of the disease gives rise to reduced level of the key neurotransmitter, dopamine in these areas.

Reduced dopamine activity gives rise to numerous symptoms, many of them extremely unpleasant and embarrassing for the sufferer. The main symptoms are an uncontrollable tremor, particularly in the limbs, which is usually worse when a limb is at rest; increased rigidity/stiffness in the limbs (“cogwheeling”); and bradykinesias (reduced/slower movements, often manifest by shuffling when walking, soft speech and swallowing difficulties). However many other symptoms have been noted, including joint and muscle pain, dribbling, postural hypotension and dizziness, in addition to dementia, which can often occur at later stages of the disease.

Similar symptoms are also known to arise secondary to other causes including as a side-effect from certain anti-psychotic and anti-nausea drugs and past encephalitis. Such secondary symptoms are usually referred to together as “parkinsonism”.

There is no known cure for Parkinson's but, fortunately, much can be done to alleviate the symptoms. In particular, the introduction of levodopa, or “L-dopa”, in the late 1960s revolutionised the treatment of the condition. L-dopa works by increasing the levels of dopamine in the affected areas of the brain in order to control directly tremors and stiffness and is still the best option for tackling the impaired motor symptoms.

Unfortunately however, L-dopa is not without its problems. In particular, although initial treatment gives rise to a dramatic alleviation of symptoms, long-term use gives rise to a notable variability in the drug's ability to control those symptoms (so-called “motor fluctuations”). Motor fluctuations may be manifest by end of dose deterioration (i.e. a sufferer noticing that the effect of his regular dose wears off prior to his scheduled time for the next dose), involuntary fidgety movements (dyskinesias) and, most disturbingly, sudden and unexpected re-appearance of symptoms, in particular stiffness, a sensation some sufferers liken to a light switch being turned on and off (so-called “on-off syndrome” of “on-off fluctuations”). All of these motor fluctuations may give rise to undesirable episodes of stiffness in a patient receiving L-dopa therapy and it is such episodes that this invention seeks to address.

As Parkinson's disease progresses, motor fluctuations become less closely associated with the timing of L-dopa dosages and more unpredictable. Such episodes are very difficult to control and attempts to manage them usually comprise increasing and/or decreasing the frequency and/or amount of L-dopa dosages and the use of L-dopa-containing controlled release formulations. However, these treatments are largely ineffective, are inconvenient, or result in exposure of the patient to higher levels of drugs than are strictly necessary to control the underlying Parkinson's symptoms In view of these difficulties, there remains a clear unmet clinical need for an effective treatment of the motor fluctuations in patients receiving L-dopa therapy.

International patent applications WO 00/16750 and WO 2004/067004 disclose drug delivery systems for the treatment of acute disorders by e.g. sublingual administration, in which the active ingredient is in microparticulate form and is adhered to the surfaces of larger carrier particles in the presence of a bioadhesive and/or mucoadhesive promoting agent. The treatment of Parkinson' disease, in particular with L-dopa, is neither mentioned nor suggested in these documents.

According to a first aspect of the invention there are provided pharmaceutical compositions that are suitable for inter alia the treatment of motor fluctuations in a patient receiving L-dopa for the treatment of Parkinson's disease comprising a weakly acidic material and a pharmacologically-effective amount of L-dopa as active ingredient, which active ingredient is presented in particulate form upon the surfaces of larger carrier particles, and which compositions are referred to hereinafter as “the compositions of the invention”.

It is preferred that the carrier particles of the compositions of the invention:
(a) comprise a weakly acidic material; and/or
(b) have (e.g. smaller) particles of a weakly acidic material presented upon the surfaces thereof; and/or
(c) have (e.g. smaller) particles of a weakly acidic material presented in between them.

The compositions of the invention are interactive mixtures. The term “interactive” mixture will be understood by those skilled in the art to denote a mixture in which particles do not appear as single units, as in random mixtures, but rather where smaller particles (of, for example, active ingredient and/or wealdy acidic material) are attached to (i.e. adhered to or associated with) the surfaces of larger carrier particles. Such mixtures are characterised by interactive forces (for example van der Waals forces, electrostatic or Coulombic forces, and/or hydrogen bonding) between carrier and surface-associated particles (see, for example, Staniforth, Powder Technol., 45, 73 (1985)). In the final mixture, the interactive forces need to be strong enough to keep the adherent particles at the carrier surface, in order to create a homogeneous mixture.

The compositions of the invention find utility in inter alia the control of motor fluctuations that are manifest by undesirable episodes of stiffness in Parkinson's patients receiving L-dopa therapy, particular those at more advanced stages of the disease. It is well known that such episodes can be sudden and unexpected and are almost always inconvenient, particularly because a patient often has a desire to be mobile when onset occurs. As described herein, the compositions of the invention may comprise a preferably small dose of active ingredient, which is released predictably and rapidly after administration for absorption e.g. via a mucosal surface for rapid, on demand relief of such symptoms.

In this respect, the term “pharmacologically effective amount” refers to an amount of active ingredient (i.e. L-dopa), which is capable of conferring the desired therapeutic effect on a treated patient (such as alleviation of motor fluctuations, in particular undesirable stiffness/rigidity episodes), whether administered alone or in combination with another active ingredient. Such an effect may be objective (i.e. measurable by some test or marker) or subjective (i.e. the subject gives an indication of, or feels, an effect).

Active ingredient is preferably presented in compositions of the invention in the form of microparticles, preferably with a weight based mean diameter of between about 0.5 μm and about 15 μm, such as about 1 μm and about 10 μm. The term “weight based mean diameter” will be understood by the skilled person to include that the average particle size is characterised and defined from a particle size distribution by weight, i.e. a distribution where the existing fraction (relative amount) in each size class is defined as the weight fraction, as obtained e.g. by sieving.

Microparticles of active ingredient may be prepared by standard micronisation techniques, such as grinding, dry milling, wet milling, precipitation, etc.

The amount of active ingredient that may be employed in compositions of the invention may be determined by the physician, or the skilled person, in relation to what will be most suitable for an individual patient. This is likely to vary with the severity of the condition that is to be treated, as well as the age, weight, sex, renal function, hepatic function and response of the particular patient to be treated.

Suitable quantities of active ingredient that may be employed in a composition of the invention may be in the range 2 to 20% by weight based upon the total weight of the composition. More preferably, compositions of the invention may contain between 4 and 17% by weight of active ingredient, and especially from about 5 to about 15%. The amount of active ingredient may also be expressed as the absolute amount in a unit dosage form (e.g. a tablet). In such a case, the total amount of active ingredient that may be present may be sufficient to provide a dose of drug per unit dosage form that is in the range about 1 to about 20 mg, such as about 2 to about 15 mg, including such as about 3 to about 13 mg and in particular between about 4 and about 12 mg.

The above-mentioned dosages are exemplary of the average case; there can, of course, be individual instances where higher or lower dosage ranges are merited, and such are within the scope of this invention.

It is possible that the relative sizes and amounts of the particles of active ingredient and the carrier particles that are employed are sufficient to ensure that the carrier particles may be at least about 90% covered by the active ingredient, for example at least about 100% and up to about 200% (e.g. between about 130% and about 180%) covered. The skilled person will appreciate in this context that “100% coverage” of the carrier particles by the active ingredient means that the relative particle sizes and amounts of the relevant particles that are employed are sufficient to ensure that the entire surface area of each carrier particle could be covered by particles of active ingredient notwithstanding that other ingredients (e.g. mucoadhesion promoting agent) may also be present in a composition. Obviously, if other such ingredients are employed, then the actual degree of coverage of carrier particles by active ingredient may be less than the amounts specified above. 200% coverage means that there is sufficient particles of active ingredient to cover the surfaces of the carrier particles twice over, notwithstanding the presence of other ingredients.

It is surprising that compositions with greater than 90% theoretical coverage are effective. Based on current knowledge, the skilled person would understand that, in order to ensure rapid dissolution, it would be important to ensure that the relative sizes/amounts of active ingredient/carrier particles are sufficient to ensure that 70% or less of the surfaces of the latter could be covered by the former.

Compositions of the invention preferably also comprise one or more bioadhesion and/or mucoadhesion promoting agent which is also presented on the surfaces of the carrier particles and, accordingly, may thus facilitate the partial or complete adhesion of active ingredient to a biological surface, such as a mucosal membrane.

The terms “mucoadhesive” and “mucoadhesion” refer to adhesion or adherence of a substance to a mucous membrane within the body, wherein mucous is present on the surface of that membrane (e.g. the membrane is substantially (e.g. >95%) covered by mucous). The terms “bioadhesive” and “bioadhesion” refer to adhesion or adherence of a substance to a biological surface in a more general sense. Biological surfaces as such may include mucous membranes wherein mucous is not present on that surface, and/or surfaces that are not substantially (e.g. <95%) covered by mucous. The skilled person will appreciate that, for example, the expressions “mucoadhesion” and “bioadhesion” may often be used interchangeably. In the context of the present invention, the relevant terms are intended to convey a material that is capable of adhering to a biological surface when placed in contact with that surface (in the presence of mucous or otherwise) in order to enable compositions of the invention to adhere to that surface. Such materials are hereinafter referred to together as “bio/mucoadhesives” or “bio/mucoadhesion promoting agents”, and such properties together as “bio/mucoadhesion” or “bio/mucoadhesive”.

A variety of polymers known in the art can be used as bio/mucoadhesion promoting agents, for example polymeric substances, preferably with an average (weight average) molecular weight above 5,000. It is preferred that such materials are capable of rapid swelling when placed in contact with water and/or, more preferably, mucous, and/or are substantially insoluble in water at room temperature and atmospheric pressure.

Bio/mucoadhesive properties may be routinely determined in a general sense in vitro, for example as described by G. Sala et al in Proceed. Int. Symp. Contr. Release. Bioact. Mat., 16, 420, 1989. Examples of suitable bio/mucoadhesion promoting agents include cellulose derivatives such as hydroxypropylmethyl cellulose (IPMC), hydroxyethyl cellulose (MEC), hydroxypropyl cellulose (HPC), methyl cellulose, ethyl hydroxyethyl cellulose, carboxymethyl cellulose, modified cellulose gum and sodium carboxymethyl cellulose NaCMC); starch derivatives such as moderately cross-linked starch, modified starch and sodium starch glycolate; acrylic polymers such as carbomer and its derivatives (Polycarbophyl, Carbopol®, etc.); polyvinylpyrrolidone; polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bio/mucoadhesive polymers can also be used.

Suitable commercial sources for representative bio/mucoadhesive polymers include: Carbopol® acrylic copolymer (BF Goodrich Chemical Co, Cleveland, 08, USA); HPMC (Dow Chemical Co., Midland, Mich., USA); NEC (Natrosol; Hercules Inc., Wilmington, Del. USA); HPC (Klucel®; Dow Chemical Co., Midland, Mich., USA); NaCMC (Hercules Inc. Wilmington, Del. USA); PEO (Aldrich Chemicals, USA); sodium alginate (Edward Mandell Co., Inc., Carmel, N.Y., USA); pectin (BF Goodrich Chemical Co., Cleveland, Ohio, USA); crosslinked polyvinylpyrrolidone (Kollidon CL®, BASF, Germany, Polyplasdone XL®, Polyplasdone XL-10® and Polyplasdone INF-10®, ISP Corp., US); Ac-Di-Sol® (modified cellulose gum with a high swellability; FMC Corp., USA); Actigum (Mero-Rousselot-Satia, Baupte, France); Satiaxana (Sanofi BioIndustries, Paris, France); Gantrez® (ISP, Milan, Italy); chitosan (Sigma, St Louis, Mo., USA); and sodium starch glycolate (Primojel®, DMV International BV, Netherlands, Vivastar®, J. Rettenmaier & Sohne GmbH & Co., Germany, Explotab®, Roquette America, US).

Preferred bio/mucoadhesion promoting agents that may be employed in compositions of the invention include internally crosslinked sodium carboxymethylcellulose, such as croscarmellose sodium NF (e.g. Ac-Di-Sol® (FMC Corp., USA)) and, particularly, crosslinked polyvinylpyrollodine (e.g. Kollidon CL®, BASF, Germany).

Depending on the type of the bio/mucoadhesion promoting agent used, the rate and intensity of bio/mucoadhesion may be varied.

Suitably, the amount of bio/mucoadhesion promoting agent that may be present in a composition of the invention may be in the range of about 0.1 to about 25% by weight based upon the total weight of the composition. A preferred range is from about 0.5 to about 15% by weight, such as about 1 to about 10% (e.g. about 2 to about 8%) by weight.

When present, bio/mucoadhesion promoting agent is at least in part presented on and/or adhered to the surface of a carrier particle in a composition of the invention.

The carrier particles may comprise, at least in part, a wealdy acidic material. When the carrier particles do not comprise a weak acid, other materials that may be employed include carbohydrates, e.g. sugar, mannitol and lactose; pharmaceutically-acceptable inorganic salts, such as sodium chloride, calcium phosphate, dicalcium phosphate hydrate, dicalcium phosphate dehydrate, tricalcium phosphate, calcium carbonate, and barium sulfate; polymers, such as microcrystalline cellulose, cellulose and crosslinked polyvinylpyrrolidone; or mixtures thereof.

In the situation when carrier particles do not comprise a weak acid, particles of the latter may be presented, at least in part, upon the surfaces of, and/or between, the former. Suitable particle sizes of weakly acid materials in such situations are as presented herein for active ingredient, bio/mucoadhesive materials and disintegrants.

In the situation when the carrier particles comprise a weak acid, such particles may consist essentially of a weak acid or may further comprise another carrier particle material as mentioned hereinbefore. In either situation, particles of weak acid may also be presented, at least in part, upon the surfaces of, and/or between, such carrier particles, as described hereinbefore. By “consisting essentially” of weak acid, we mean that, excluding the possible presence of water (vide infra), the carrier particles comprise at least about 95%, such as at least about 98%, more preferably greater than about 99%, and particularly at least about 99.5% by weight (based on the total weight of the carrier particle) of such an acid. These percentages exclude the presence of trace amounts of water and/or any impurities that may be present in such materials, which impurities may arise following the production of such materials, either by a commercial or non-commercial third party supplier, or by a skilled person making a composition of the invention.

Weakly acidic materials that may be mentioned include those that enable the provision at the site of absorption upon administration of a pH of between about 5.5 and about 6.5. For the purpose of this invention, the term includes substances that are safe for use in mammals, and includes weak acids, weak acid derivatives and other chemicals that convert to weak acids in vivo (e.g. precursors that convert to acids in vivo, by for example being sequentially activated in accordance with properties of the local environment). More preferably, the weakly acidic material comprises a weak acid that is safe for human consumption, for example a food acid, such as citric acid, tartaric acid, amalic acid, fumeric acid, adipic acid, succinic acid or a combination thereof.

Preferably, carrier particles for use in compositions of the invention are of a size that is between about 50 and about 750 μm, and preferably between about 100 and about 600 μm.

Compositions of the invention, once prepared, are preferably directly compressed/compacted into unit dosage forms (e.g. tablets) for administration to mammalian (e.g. human) patients, for example as described hereinafter.

A disintegrating agent, or “disintegrant” may also be included in the composition of the invention, particularly those that are in the form of tablets for e.g. sublingual administration. Such an agent may be defined as any material that is capable of accelerating to a measurable degree the disintegration/dispersion of a composition of the invention, and in particular carrier particles, as defined herein. This may be achieved, for example, by the material being capable of swelling and/or expanding when placed in contact with water and/or mucous (e.g. saliva), thus causing tablet formulations/carrier particles to disintegrate when so wetted.

Suitable disintegrants include cross-linked polyvinylpyrrolidone, carboxymethyl starch and natural starch and mixtures thereof.

If present, disintegrating agent is preferably employed in an amount of between 0.5 and 10% by weight based upon the total weight of the composition. A preferred range is from 1 to 8%, such as from about 2 to about 7% (e.g. about 5%) by weight.

It will be evident from the list of possible disintegrants provided above that certain materials may function in compositions of the invention in the form of tablets both as bio/mucoadhesion promoting agents and as disintegrating agents. Thus, these functions may both be provided by different substances or may be provided by the same substance.

When the “same” material is employed as a bio/mucoadhesive and as a disintegrant, the material can be said to be in two separate fractions (a bio/mucoadhesive fraction and a disintegrant fraction). In such instances, it is preferred that the particles within the disintegrant fraction are coarser (i.e. are, relatively speaking, of a larger particle size) than those in the bioadhesive fraction (vide infra).

In any event, the skilled person will appreciate that, in compositions of the invention in the form of tablets, any disintegrant (or disintegrant fraction) will be largely not presented on (i.e. attached to, adhered to and/or associated with) the surfaces of the carrier particles, but rather will be largely presented (i.e. at least about 60%, such as about 70%, e.g. about 80% and, more particularly, about 90% by weight presented) between such particles. Conversely, bio/mucoadhesive (or bio/mucoadhesive fraction) is always largely associated (i.e. is at least about 60%, such as about 70%, e.g. about 80% and, more particularly, about 90% by weight associated) with the carrier particles, that is to say presented on (i.e. attached to, adhered to and/or associated with) the surfaces of the carrier particles, or presented within such particles (vide infira), or both.

Compositions of the invention in the form of tablets for e.g. sublingual administration may also comprise a binder. A binder may be defined as a material that is capable of acting as a bond formation enhancer, facilitating the compression of the powder mass into coherent compacts. Suitable binders include cellulose gum and microcrystalline cellulose. If present, binder is preferably employed in an amount of between 0.5 and 20% by weight based upon the total weight of the tablet formulation. A preferred range is from 1 to 15%, such as from about 2.0 to about 12% (e.g. about 10%) by weight.

Compositions of the invention may comprise a pharmaceutically acceptable surfactant or wetting agent, which may enhance the hydration of active ingredient and carrier particles, resulting in faster initiation of both bio/mucoadhesion and dissolution. If present, the surfactant should be provided in finely dispersed form and mixed intimately with the active ingredient. Examples of suitable surfactants include sodium lauryl sulphate, lecithin, polysorbates, bile acid salts and mixtures thereof. If present, the surfactant may comprise between about 0.3 and about 5% by weight based upon the total weight of the composition, and preferably between about 0.5 and about 3% by weight.

Suitable further additives and/or excipients that may be employed in compositions of the invention, in particular those in the form of tablets for e.g. sublingual administration may comprise:
(a) lubricants (such as sodium stearyl fumarate or, preferably, magnesium stearate). When a lubricant is employed it should be used in very small amounts (e.g. up to about 3%, and preferably up to 2%, by weight based upon the total weight of the tablet formulation);
(b) flavourings (e.g. lemon, menthol or, preferably, peppermint powder), sweeteners (e.g. neohesperidin) and dyestuffs;
(c) antioxidants, which may be naturally occurring or otherwise (e.g. vitamin C, vitamin E, β-carotene, uric acid, uniquion, SOD, glutathione peroxidase or peroxidase catalase);
(d) other ingredients, such as carrier agents, preservatives and gliding agents; and/or
(e) a dopamine decarboxylase inhibitor (e.g. carbidopa or benserazide), which may be given in combination with L-dopa to increase the amount of active medication available for pharmacological action, and/or to prevent dopamine from building up in the body (in particular the stomach), thereby reducing unwanted side effects such as nausea and vomiting.

Compositions of the invention may be prepared by standard techniques, and using standard equipment, known to the skilled person.

For example, if present, bio/mucoadhesion promoting agent and/or particles of weakly acidic material may be admixed with carrier particles in several ways. In one embodiment, bio/mucoadhesion promoting agent, and/or weakly acidic material, in fine particulate form is/are mixed together with coarse carrier for a sufficient time in order to produce an ordered or interactive mixture. This results in discrete particles of bio/mucoadhesion promoting agent, and/or weakly acidic material, being presented on and/or adhered to the surfaces of the carrier particles. The skilled person will appreciate that, in order to obtain a dry powder formulation in the form of an interactive mixture, larger carrier particles must be able to exert enough force to break up agglomerates of smaller particles. This ability will primarily be determined by particle density, surface roughness, shape, flowability and, particularly, relative particle sizes.

If present, the bio/mucoadhesion promoting agent suitably has a particle size with a weight based mean diameter of between about 0.1 and about 100 μm (e.g. about 1 and about 50 μm).

Active ingredient may be dry mixed with carrier particles over a period of time that is sufficiently long to enable appropriate amounts of active ingredient to adhere to the surface of the carrier particles (with or without the presence of bio/mucoadhesion promoting agent). Standard mixing equipment may be used in this regard. The mixing time period is likely to vary according to the equipment used, and the skilled person will have no difficulty in determining by routine experimentation a suitable mixing time for a given combination of active ingredient and carrier particle material.

Other ingredients (e.g. disintegrants and surfactants) may be incorporated by standard mixing as described above for the inclusion of active ingredient.

The compositions of the invention may be administered transmucosally, such as buccally, rectally, nasally or preferably sublingually by way of appropriate dosing means known to the skilled person. A sublingual tablet may be placed under tongue, and the active ingredient absorbed through the surrounding mucous membranes.

In this respect, the compositions of the invention may be incorporated into various kinds of pharmaceutical preparations intended for transmucosal (e.g. sublingual) administration using standard techniques (see, for example, Lachman et al, “The Theory and Practice of Industrial Pharmacy”, Lea & Febiger, 3^{rd} edition (1986) and “Remington: The Science and Practice of Pharmacy”, Gennaro (ed.), Philadelphia College of Pharmacy & Sciences, 19^{th} edition (1995)).

Pharmaceutical preparations for sublingual administration may be obtained by combining compositions of the invention with conventional pharmaceutical additives and/or excipients used in the art for such preparations, and thereafter preferably directly compressed/compacted into unit dosage forms (e.g. tablets). (See, for example, Pharmaceutical Dosage Forms. Volume 1, 2^{nd} Edition, Lieberman et al (eds.), Marcel Dekker, New York and Basel (1989) p. 354-356 and the documents cited therein.) Suitable compacting equipment includes standard tabletting machines, such as the Kilian SP300 or the Korsch EK0.

Suitable final sublingual tablet weights are in the range 30 to 400 mg, such as 50 to 200 mg, for example 60 to 180 mg, more preferably between about 70 and about 160 mg. Suitable final tablet diameters are in the range 4 to 10 mm, for example 5 to 9 mm, and more preferably about 6 to about 8 mm.

Irrespective of the foregoing, if a composition of the invention comprises a bio/mucoadhesion promoting agent, it should be essentially free (e.g. less than about 20% by weight based on the total weight of the formulation) of water. It will be evident to the skilled person that “premature” hydration will dramatically decrease the mucoadhesion promoting properties of such a tablet formulation and may result in premature dissolution of the active ingredient.

Wherever the word “about” is employed herein in the context of dimensions (e.g. tablet sizes and weights, particle sizes, pH values etc.), surface coverage (e.g. of carrier particles by active ingredient), amounts (e.g. relative amounts of individual constituents in a composition or a component of a composition and absolute doses of active ingredient), it will be appreciated that such variables are approximate and as such may vary by ±10%, for example ±5% and preferably ±2% (e.g. ±1%) from the numbers specified herein.

Compositions of the invention may be administered by way of appropriate dosing means known to the skilled person. For example, a sublingual tablet may be placed under the tongue, and the active ingredient absorbed through the surrounding mucous membrane.

The compositions of the invention are useful in the treatment of Parkinson's disease and in particular the symptomatic treatment of motor fluctuations, such as the undesirable stiffness episodes mentioned hereinbefore, in patients receiving L-dopa for the treatment of Parkinson's disease. The term “Parkinson's disease” also includes, for the purposes of this invention, so-called parkinsonism and diseases that are or may be treated by L-dopa. According to a farther aspect of the invention there is provided a method of treatment of motor fluctuations in a patient receiving L-dopa for the treatment of Parkinson's disease which method comprises administration of a composition of the invention to a person suffering from, or susceptible to, such fluctuations.

For the avoidance of doubt, by “treatment” we include the therapeutic treatment, as well as the symptomatic treatment, the prophylaxis, or the diagnosis, of a condition.

Also disclosed herein are compositions in which the inclusion of bio/mucoadhesion promoting agent is an essential feature. In such instances, the use of wealdy acidic material as, attached to, and/or between, the carrier particles is inessential. Apart from these differences, all other features of the compositions of the invention described herein are equally applicable to such compositions.

The compositions of the invention enable the production of unit dosage forms that are easy and inexpensive to manufacture, and which enable the rapid release and/or a rapid uptake of active ingredient through the mucosa, such as the oral mucosa, thus enabling rapid relief of the symptoms described hereinbefore.

The compositions of the invention may also have the advantage that they substantially reduce the degree of absorption of active ingredient via swallowed saliva, as well as enabling the administration of “reduced” amounts of the active ingredient that is employed, so substantially reducing the risk of side effects, as well as intra- and interpatient variability of therapeutic response.

Compositions of the invention may also have the advantage that they may be prepared using established pharmaceutical processing methods and employ materials that are approved for use in foods or pharmaceuticals or of like regulatory status.

Compositions of the invention may also have the advantage that they may be more efficacious than, be less toxic than, be longer acting than, be more potent than, produce fewer side effects than, be more easily absorbed than, and/or have a better pharmacokinetic profile than, and/or have other useful pharmacological, physical, or chemical properties over, pharmaceutical compositions known in the prior art, whether for use in the treatment of Parkinson's disease or otherwise.

The invention is illustrated by way of the following examples.

### EXAMPLE 1

L-dopa (Fluka, Switzerland) is firstly micronised and then accurately weighed out, along with the other excipients (see below), in appropriate proportions that enable the production of tablets with the absolute amounts of various ingredients mentioned below.

Pre-weighed quantities of L-dopa and citric acid are then mixed in a Turbula mixer for 96 hours. Then, pre-weighed quantities of silicified microcrystalline cellulose (ProSolv; JRS Pharma, Germany) and sodium carboxymethylcellulose (Croscarmellose Sodium NF; Ac-Di-Sol®; FMC Corp., USA) are added and mixing is continued for 30 minutes. Finally, a pre-weighed quantity of magnesium stearate (Peter Greven, Netherlands) is added and mixing continued for another 2 minutes.

The powder mixture is then compacted using a single punch press (Korsch EK0) with 6 mm flat bevel edged punches, to produce tablets of a total weight of 100 mg.

The absolute amounts of individual ingredients are as presented in the table below.

In-process controls are employed (tablet weight, crushing strength, friability and disintegration time), with test samples being withdrawn throughout the tabletting process. Tablets are packaged and labelled.

| Ingredient | Amount (mg) |
|---|---|
| L-dopa | 5.00 |
| citric acid | 50.00 |
| silicified microcrystalline cellulose | 4.00 |
| sodium carboxymethylcellulose | 40.00 |
| magnesium stearate | 1.00 |
| Total tablet weight | 100.00 |

### EXAMPLE 2

A tablet composition is prepared in accordance with the procedure described in Example 1 above, with mannitol (Roquette, FR) being added in the first mix. The absolute amounts of individual ingredients are presented in the table below.

| Ingredient | Amount (mg) |
|---|---|
| L-dopa | 5.00 |
| citric acid | 10.00 |
| mannitol | 40.00 |
| silicified microcrystalline cellulose | 4.00 |
| sodium carboxymethylcellulose | 40.00 |
| magnesium stearate | 1.00 |
| Total tablet weight | 100.00 |

### EXAMPLE 3

L-dopa (Fluka, Switzerland) and carbidopa (Sigma-Aldrich, USA) were firstly micronised and then accurately weighed out as described in Example 1.

Pre-weighed quantities of L-dopa, carbidopa and mannitol (Mannitol 400 DC; Roquette, France) were then mixed in a mixer for 96 hours. Then, pre-weighed quantities of citric acid (Roche, Belgium), silicified microcrystalline cellulose (ProSolv; Penwest Pharmaceutical Co, USA) and sodium carboxymethylcellulose (Croscarmellose Sodium NF; Ac-Di-Sol®; FMC Corp., USA) were added and mixing was continued for 30 minutes. Finally, a pre-weighed quantity of magnesium stearate (Peter Greven, Netherlands) was added and mixing continued for another 2 minutes.

The powder mixture was then compacted using a single punch press (Korsch EK0) with 6 mm flat bevel edged punches, to produce tablets of a total weight of 95.1 mg.

The absolute amounts of individual ingredients are as presented in the table below. In-process controls were employed, and tablets were packaged and labelled as described in Example 1.

| Ingredient | Amount (mg) |
|---|---|
| L-dopa | 5.20 |
| carbidopa | 1.20 |
| citric acid | 19.90 |
| mannitol | 55.20 |
| silicified microcrystalline cellulose | 8.90 |
| sodium carboxymethylcellulose | 4.00 |
| magnesium stearate | 0.70 |
| Total tablet weight | 95.10 |

### EXAMPLE 4

L-dopa and carbidopa were micronised and weighed out as described in Example 3.

Pre-weighed quantities of L-dopa, carbidopa, citric acid and mannitol were mixed as described in Example 3 for 96 hours. Then, pre-weighed quantities of silicified microcrystalline cellulose and sodium carboxymethylcellulose were added and mixing continued as described in Example 3 for 30 minutes. Finally, pre-weighed magnesium stearate was added and mixing continued for another 2 minutes.

Tablets were produced as described in Example 3 with absolute amounts of individual ingredients as presented in the table below.

| Ingredient | Amount (mg) |
|---|---|
| L-dopa | 5.00 |
| carbidopa | 1.30 |
| citric acid | 6.60 |
| mannitol | 57.30 |
| silicified microcrystalline cellulose | 7.00 |
| sodium carboxymethylcellulose | 3.10 |
| magnesium stearate | 0.40 |
| Total tablet weight | 80.70 |

1. A pharmaceutical composition comprising a weakly acidic material and a pharmacologically-effective amount of L-dopa as active ingredient, which active ingredient is presented in particulate form upon the surfaces of larger carrier particles.

2. A composition as claimed in claim 1, wherein at least one of the following applies:
(a) the carrier particles comprise the weakly acidic material; and/or
(b) particles of the weakly acidic material are presented upon the surfaces of the carrier particles; and/or
(c) particles of the weakly acidic material are presented between the carrier particles.

3. A composition as claimed in claim 1 or claim 2, wherein the active ingredient is in the form of microparticles.

4. A composition as claimed in claim 3, wherein the microparticles have a weight based mean diameter of less than about 15 μm.

5. A composition as claimed in claim 1, wherein the total amount of active ingredient present is in the range about 2 to about 20% by weight based upon the total weight of the composition.

6. A composition as claimed in claim 5, wherein the range is about 5 to about 15% by weight.

7. A composition as claimed in claim 1, which further comprises a bioadhesion and/or mucoadhesion promoting agent.

8. A composition as claimed in claim 7, wherein the bioadhesion and/or mucoadhesion promoting agent is a polymeric substance with a weight average molecular weight above 5,000.

9. A composition as claimed in claim 8, wherein the bioadhesion and/or mucoadhesion promoting agent is selected from a cellulose derivative, a starch derivative, an acrylic polymer, polyvinylpyrrolidone, polyethylene oxide, chitosan, a natural polymer, scleroglucan, xanthan gum, guar gum, poly co-(methylvinyl ether/maleic anhydride) and crosscarmellose, or a mixture thereof.

10. A composition as claimed in claim 9, wherein the bioadhesion and/or mucoadhesion promoting agent is selected from hydroxypropylmethyl cellulose, hydroxyethyl cellulose, hydroxypropyl cellulose, methyl cellulose, ethyl hydroxyethyl cellulose, carboxymethyl cellulose, modified cellulose gum, sodium carboxymethyl cellulose, moderately cross-linked starch, modified starch, sodium starch glycolate, carbomer or a derivative thereof, crosslinked polyvinylpyrrolidone, polyethylene oxide, chitosan, gelatin, sodium alginate, pectin, scleroglucan, xanthan gum, guar gum, poly co-(methylvinyl ether/maleic anhydride) and crosscarmellose sodium, or a mixture thereof.

11. A composition as claimed in claim 10, wherein the bioadhesion and/or mucoadhesion promoting agent is crosscarmellose sodium or crosslinked polyvinylpyrrolidone.

12. A composition as claimed in claim 11, wherein the amount of bioadhesion and/or mucoadhesion promoting agent present is in the range of about 0.1 to about 25% by weight based upon the total weight of the composition.

13. A composition as claimed in claim 12, wherein the range is about 1 to about 15% by weight.

14. A composition as claimed in claim 1 wherein the carrier particles comprise a weakly acidic material.

15. A composition as claimed in claim 14, wherein particles of weakly acidic material are also presented, at least in part, upon the surfaces of the carrier particles.

16. A composition as claimed in claim 1 wherein the carrier particles do not comprise a weakly acidic material, and particles of weakly acidic material are presented upon the surfaces of the carrier particles.

17. A composition as claimed in claims claim 1, wherein particles of weakly acidic material are presented between the carrier particles.

18. A composition as claimed in any one of the preceding claims, wherein the carrier particles comprise or include a carbohydrate, a pharmaceutically-acceptable inorganic salt, a polymer or a mixture thereof.

19. A composition as claimed in claim 18, wherein the particles comprise or include sugar, mannitol, lactose, sodium chloride, calcium phosphate, dicalcium phosphate hydrate, dicalcium phosphate dehydrate, tricalcium phosphate, calcium carbonate, barium sulfate, microcrystalline cellulose, cellulose, crosslinked polyvinylpyrrolidone or a mixture thereof.

20. A composition as claimed in claim 19, wherein the particles comprise or include mannitol and/or lactose.

21. A composition as claimed in claim 1 wherein the weakly acidic material is a food acid.

22. A composition as claimed in claim 21, wherein the acid is citric acid, tartaric acid, amalic acid, fumeric acid, adipic acid, succinic acid or a combination thereof.

23. A composition as claimed in claim 22, wherein the acid is citric acid.

24. A composition as claimed in claim 1, wherein the carrier particle size is between about 50 and about 750 μm.

25. A composition as claimed in claim 24, wherein the particle size is between about 100 and about 600 Tm.

26. A composition as claimed in claim 7 wherein the bioadhesion and/or mucoadhesion promoting agent has a particle size in the range of about 1 to about 100 Tm.

27. A composition as claimed in claim 1, wherein the relative sizes and amounts of the particles of active ingredient and the carrier particles that are employed are sufficient to ensure that the carrier particles may be at least about 90% covered by the active ingredient.

28. A composition as claimed in claim 1, which further comprises a dopamine decarboxylase inhibitor.

29. A composition as claimed in claim 1, which is in the form of a tablet suitable for sublingual administration.

30. A composition as claimed in claim 29, wherein the composition further comprises a disintegrating agent.

31. A composition as claimed in claim 30, wherein the disintegrating agent is selected from crosslinked polyvinylpyrrolidone, carboxymethyl starch, natural starch and mixtures thereof.

32. A composition as claimed in claim 30 or claim 31, wherein the amount of disintegrating agent is between about 2 and about 7% by weight based upon the total weight of the composition.

33. A process for the preparation of a composition as defined in claim 1, which comprises dry mixing carrier particles together with the active ingredient and the further particles of weak acid (if present).

34. A process as claimed in claim 33 wherein bioadhesion and/or mucoadhesion promoting agent is also mixed together in fine particulate form with carrier particles.

35. A process for the preparation of a sublingual tablet, which comprises directly compressing or compacting a composition as defined in claim 1.

36. (canceled)

37. A method of treatment of Parkinson's disease which method comprises administration of a composition as defined in claim 1 to a patient suffering from, or susceptible to, such a condition.

38. A method as claimed in claim 37, wherein the treatment is of motor fluctuations in patients receiving L-dopa for the treatment of Parkinson's disease.
